(19) United States
(12) Patent Application Publication
(10) Pub. No.: US 2025/0207190 A1
(43) Pub. Date: Jun. 26, 2025

(54) Method for Detecting Acute Rejection, Defining Therapy and Monitoring Rejection Therapy

(71) Applicant: University of Cincinnati, Cincinnati, OH (US)

(72) Inventors: David Hildeman, Cincinnati, OH (US); Ervin Steve Woodle, Liberty Township, OH (US)

(73) Assignee: University of Cincinnati, Cincinnati, OH (US)

(21) Appl. No.: 19/000,382

(22) Filed: Dec. 23, 2024

Related U.S. Application Data
(60) Provisional application No. 63/614,424, filed on Dec. 22, 2023.

(51) Int. Cl. C12Q 1/6869; C12Q 1/6806

(52) U.S. Cl. CPC C12Q 1/6869; C12Q 1/6806; G01N 2800/245; G01N 2800/52

(57) ABSTRACT

A method for performing an analysis related to potential rejection of an organ after transplantation in a subject is provided. The method involves identifying alloreactive/expanded CD8 T cell clones in either an allograft biopsy or a urine sample from the subject, wherein the T cell clones are identified using a single cell RNASeq (scRNAseq)/TCRseq test. In one embodiment, the analysis is selected from the group consisting of detection of rejection, diagnosis of rejection, treatment selection, therapeutic monitoring for rejection, identification of resistance to rejection treatment, and combinations thereof.

### CROSS-REFERENCE TO RELATED APPLICATIONS

This application claims priority to, and the benefit of the filing date of, U.S. Provisional Application No. 63/614,424 filed Dec. 22, 2023, the disclosure of which is incorporated by reference herein in its entirety.

### STATEMENT REGARDING FEDERALLY FUNDED RESEARCH OR DEVELOPMENT

This invention was made with government support under AI154932 awarded by the National Institutes of Health. The government has certain rights in the invention.

### BACKGROUND OF THE INVENTION

This section is intended to introduce the reader to various aspects of the art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Rejection remains the major cause that kidney, heart, lung, pancreas, liver and small intestinal transplants are lost. Moreover, the treatments for rejection are over 70 years old and are empirically rather than mechanistically based. Development of approaches for detecting rejection, monitoring therapy, and determining mechanistically based treatments are lacking and sorely needed. Advancements in these areas will be of significant importance in improving survival after transplantation and mitigating the organ shortage problem by prolonging transplant survival.

Previous studies of acute rejection have indicated three major clinical phenotypes: acute cellular rejection (ACR), antibody mediated rejection (AMR), and mixed acute rejection (MAR) which is defined as coexisting ACR and AMR. Banff criteria have been established by collaborative efforts of clinicians and pathologists to provide a standardized means for diagnosing and stratifying rejection episodes according to phenotype and severity (REF). Although molecular approaches consisting of gene expression data derived from bulk transcriptomic analysis (using gene chips (Affymetrix)) of renal allograft biopsies using the Molecular Microscope (REF) platform, the Molecular Microscope has significant limitations due to substantial overlap in gene expression between the varying clinical phenotypes of rejection. As such, the Molecular Microscope has not gained widespread adoption. Other approaches have been attempted using peripheral blood as a means to molecularly phenotype rejection, and these also have not proven clinically reliable, likely due to the failure of peripheral blood-based analyses to accurately reflect intragraft rejection events. Finally, the more recent development of donor derived cell free DNA (ddcfDNA) has also proven to be unable to distinguish between AMR and ACR, and importantly, is relatively insensitive for diagnosing ACR. ddcfDNA assays also do not provide information on the nature of rejection, nor can they provide information that can provide a basis for therapeutic selection.

Analysis of rejection episodes of all clinical phenotypes (early and late ACR or AMR or MAR) have provided important information regarding long term allograft survival according to clinical phenotype. Other than early Banff 1a ACR rejection episodes, which have good 3-5 year survival with high dose corticosteroid therapy (ie, less than 10% graft loss at 3 years), other more severe rejections, such as Banff 2a or 2b have less than 50% graft survival at 3-5 years (REF). Importantly, the presence of either AMR or a donor specific antibody (DSA) at the time of rejection also confers a significant risk of accelerated graft loss. Also, late rejections, as compared by each individual Banff class to early rejections (ie, occurring within 6 months posttransplant) are associated with much worse graft loss rates. Collectively, these data show that the options for treating high severity rejections are woefully inadequate.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention.

In an embodiment of the invention, a method for performing an analysis related to potential rejection of an organ after transplantation in a subject is provided. The method involves identifying alloreactive/expanded CD8 T cell clones in either an allograft biopsy or a urine sample from the subject, wherein the T cell clones are identified using a single cell RNASeq (scRNAseq)/TCRseq test. In one embodiment, the analysis is selected from the group consisting of detection of rejection, diagnosis of rejection, treatment selection, therapeutic monitoring for rejection, identification of resistance to rejection treatment, and combinations thereof.

In another embodiment, alloreactive/expanded CD8 T cell clones are identified from an allograft biopsy. In one embodiment, alloreactive/expanded CD8 T cell clones are identified from a urine sample. In another embodiment, the analysis comprises detecting rejection of a transplanted organ in a subject. In one embodiment, the analysis comprises monitoring for rejection of a transplanted organ in a subject. In another embodiment, the analysis comprises monitoring rejection therapy.

In one embodiment, the organ is a kidney. In another embodiment, the allograft biopsy is a renal allograft biopsy. In one embodiment, the renal allograft biopsy involves tissue digestion with cold digestion proteases.

In another embodiment of the invention, a method for assessing responses of a subject to anti-rejection therapies is provided. The method involves identifying alloreactive/expanded CD8 T cell clones in either an allograft biopsy or a urine sample from the subject, wherein the T cell clones are identified using a single cell RNASeq (scRNAseq)/TCRseq test.

In another embodiment of the present invention, a method for identifying specific cell populations that are causing rejection of a transplanted organ in a subject is provided. The method involves identifying alloreactive/expanded CD8 T cell clones in either an allograft biopsy or a urine sample from the subject, wherein the T cell clones are identified using a single cell RNASeq (scRNAseq)/TCRseq test. In one embodiment, the specific cell populations are CD8+ T cells that express effector molecules. In another embodiment, the CD8+ T cells are selected from the group consisting of granzymes, perforin and combinations thereof. In one embodiment, the method further involves using transcriptomic profiling on the specific cell populations to determine signaling pathways and analyzing the signaling pathways using pathway identification software.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The objects and advantages of the present invention will be further appreciated in light of the following detailed descriptions and drawings in which:

FIG. 1 is a schematic showing results from scRNAseq analysis of 10 renal allograft biopsies including three biopsies from patients on tacrolimus-based immunosuppression who were not experiencing rejection, and 4 from patients on tacrolimus-based immunosuppression that were experiencing rejection, 3 from patients on belatacept (CD28 blockade) based immunosuppression that were experiencing rejection, and 3 from patients on iscalimab (CD40 blockade) based immunosuppression that were experiencing rejection.

FIG. 2A is a UMAP plot that displays cell contribution by sample and cell type.

FIG. 2B is another UMAP plot that displays cell contribution by sample and cell type.

FIG. 2C is a series of graphs showing expression of “signature” genes across cell types. Blue color intensity reflects the expression level of individual genes within given cells.

FIG. 2D is a UMAP plot showing separation of samples based on rejection status. UMAP plots show cells from no-rejection samples (gray, left plot) versus rejection samples (pink, right plot).

FIG. 2E is a graph showing frequency of cell types within each sample displayed in bar graphs. Statistical analyses reveal a significantly increased proportion of immune infiltration in the rejection samples (n=10) compared with the no-rejection samples (n=3). Two-tailed t test, ****p<0.0001.

FIG. 3A is a UMAP plot showing immune-cell clusters and accompanying annotations.

FIG. 3B is a series of violin plots displaying the relative gene expression levels of indicated genes across each cluster.

FIG. 3C is a series of UMAP plots showing immune-cell clustering of samples from participants with rejection under tacrolimus (left plot, shades of mustard), belatacept (middle plot, shades of blue), or iscalimab (right plot, shades of pink) maintenance IS. Samples were segregated according to maintenance IS type.

FIG. 3D is a series of graphs showing the frequency of cell types within each sample displayed in bar graphs. Statistical analyses revealed a significantly increased proportion of CD8⁺ T cells in the immune infiltration as compared with other immune subtypes (n=10). One-way ANOVA. ***p<0.0006; **p<0.007.

FIG. 4A is a UMAP plot showing cell-type annotations based on DEGs.

FIG. 4B is a series of violin plots showing relative expression levels of indicated genes selected to characterize cell-cluster phenotypes as activated, exhausted, and memory.

FIG. 4C is a series of pie charts displaying number and frequency of CD8_{EXP} found in the biopsy during rejection by participant sample, based on their unique CDR3α/β sequences. Expanded clonotypes are defined as having more than 2 cells with identical CDR3α/β sequences. Different colors represent individual expanded clonotypes (gray area represents unexpanded clonotypes), and the sizes of the colored areas represent the relative sizes of the expanded clonotypes.

FIG. 4D is a pair of graphs showing percentages (left graph) and total numbers (right graph) of CD8_{EXP} in each treatment group (tacrolimus, n=4; belatacept, n=3; iscalimab, n=3) are displayed in the bar graphs (±SD). One-way ANOVA; NS, p>0.05.

FIG. 4E is a graph showing full-length TCRs with unique CDR3α/β sequences derived from 5 CD8_{EXP} from 1 participant experiencing rejection (ISCAL_1) were subcloned into individual Jurcat 76 cells. Individual clones were cultured in triplicate either alone or with donor or third-party T cell-depleted PBMCs for 20 hours and IL-2 levels in the supernatant measured via ELISA. Results show the levels of IL-2 in pg/ml for each condition (±SD) done in triplicate (n=3). One-way ANOVA. *p<0.05.

FIG. 5A is a series of UMAP plots showing clustering of CD8_{EXP} based on maintenance IS type. UMAP plots show clustering of CD8_{EXP} (colored dots) versus CD8_{UNEXP} (gray dots) from participants under either tacrolimus (left plot, shades of mustard), belatacept (right plot, shades of blue), or iscalimab (middle plot, shades of pink) maintenance IS.

FIG. 5B is a series of bar graphs displaying the fraction of expanded clonotypes (tacrolimus, belatacept, or iscalimab) and unexpanded clonotypes contributing to each CD8⁺ T cell cluster.

FIG. 5C is a pair of violin plots showing the relative expression of indicated genes in CD8_{EXP} and CD8_{UNEXP}.

FIG. 5D is a heatmap displaying (average) expression of unsupervised DEGs (p<0.05) in CD8_{EXP} under tacrolimus (n=4), belatacept (n=3), and iscalimab (n=3) maintenance IS. Blue text denotes 3 TNF family member genes, and red text denotes FKBPIA, a target of tacrolimus.

FIG. 5E is a heatmap displaying a supervised analysis of the average expression of mTOR pathway-related genes in CD8_{EXP} from participants under tacrolimus, belatacept, and iscalimab maintenance IS.

FIG. 6A is a series of pie charts displaying number and frequency of expanded clonotypes found in the index biopsy (PTD 217) and subsequent follow-up biopsies (PTD 232, PTD 295). Bar graph shows overlapping clonotypes across the three time points.

FIG. 6B is a UMAP showing CD8⁺ clusters in an integrated analysis of all time points.

FIG. 6C is a series of violin plots showing relative expression levels of indicated genes selected to characterize cell cluster phenotypes as activated, exhausted and memory.

FIG. 6D is a series of UMAP plots show clustering of CD8_{EXP} (colored dots) versus CD8_{UNEXP} (gray dots) from the participant at PTD 217 (left plot), PTD 232 (middle plot), or PTD 295 (right plot). CD8_{EXP} first expanded on PTD 217 are shown in pink, those first expanded on PTD 232 are shown in green, and first expanded on PTD 295 are shown in blue. This shows a temporal analysis of CD8_{EXP} following antirejection therapy.

FIG. 6E is a heatmap showing average expression of unsupervised DEGs (p<0.05) found between CD8_{EXP} at each time point.

FIG. 7A is a pair of pie charts displaying number and frequency of expanded clonotypes found in the index biopsy (PTD 111) and subsequent follow-up biopsy (PTD 125). Bar graph shows overlapping clonotypes across the two time points.

FIG. 7B is a UMAP showing CD8⁺ clusters in an integrated analysis of both time points.

FIG. 7C is a series of violin plots showing relative expression levels of indicated genes selected to characterize cell cluster phenotypes as activated, exhausted and memory.

FIG. 7D is a pair of UMAP plots showing clustering of CD8_{EXP} (colored dots) versus CD8_{UNEXP} (gray dots) from the participant at PTD 111 (left plot) and PTD 125 (right plot). CD8_{EXP} first expanded on PTD 111 are shown in pink, and those first expanding on PTD 125 are shown in blue. This shows a temporal analysis of CD8_{EXP} following antirejection therapy.

FIG. 7E is a heatmap showing average expression of unsupervised DEGs found between CD8_{EXP} at each time point.

FIG. 8A is a series of pie charts displaying number and frequency of expanded clonotypes found in the index biopsy (PTD 60) and subsequent follow-up biopsies (PTD 78, PTD 336). A bar graph shows overlapping clonotypes across the three time points.

FIG. 8B is a UMAP showing CD8⁺ clusters in an integrated analysis of all time points.

FIG. 8C is a series of violin plots showing relative expression levels of indicated genes selected to characterize cell cluster phenotypes as activated, exhausted and memory.

FIG. 8D is a series of UMAP plots showing clustering of CD8_{EXP} (colored dots) versus CD8_{UNEXP} (gray dots) from the participant at PTD 60 (left plot), PTD 78 (middle plot), or PTD 336 (right plot). CD8_{EXP} emerging on PTD 60 are shown in pink, those emerging on PTD 78 are shown in green, and those emerging on PTD 336 are shown in blue. This shows a temporal analysis of CD8_{EXP} following antirejection therapy.

FIG. 8E is a heatmap showing average expression of unsupervised DEGs (p<0.05) found between CD8_{EXP} at each time point.

FIG. 9A is a series of pie charts (top) displaying number and frequency of expanded clonotypes found at each biopsy (A) and urine (B) sample, and a bar graph (bottom) that displays clonotypes found at the indicated time points. Different colors represent individual expanded clonotypes (gray area represents unexpanded clonotypes), and the size of the colored area represents the relative size of the expanded clonotypes.

FIG. 9B is a series of pie charts (top) displaying number and frequency of expanded clonotypes found at each biopsy (A) and urine (B) sample, and a bar graph (bottom) that displays clonotypes found at the indicated time points. Different colors represent individual expanded clonotypes (gray area represents unexpanded clonotypes), and the size of the colored area represents the relative size of the expanded clonotypes.

FIG. 9C is a series of venn diagrams displaying overlap of individual CD8_{EXP} clonotypes between biopsies and their paired urine sample at the indicated time points.

FIG. 10A is a UMAP plot showing individual CD8⁺ clusters based on DEGs. Allograft-derived CD8⁺ T cells from all time points from participant ISCAL_3 were integrated and renormalized. Note that some clusters are unique to individual time points.

FIG. 10B is a series of violin plots showing relative expression levels of indicated genes selected to characterize cell cluster phenotypes as activated, exhausted and memory.

FIG. 10C is a series of UMAP plots showing clustering of CD8_{EXP} (colored dots) versus CD8_{UNEXP} (gray dots) from the participant at PTD 137 (left plot), PTD 151 (middle left plot), PTD 179 (middle right plot), or PTD 291 (right plot). CD8_{EXP} emerging on PTD 137 are shown in pink, on PTD 151 are shown in green, on PTD 179 are shown in blue, and those emerging on PTD 291 are shown in purple. This shows a temporal analysis of CD8_{EXP} during treatment-refractory rejection therapy.

FIG. 10D is a heatmap showing average expression of unsupervised DEGs found between expanded clonotypes at each time point.

FIG. 11A is a pair of UMAP plots illustrating the integration and clustering of CD8+ T cells from the biopsy and corresponding urine sample, both obtained at the time of ACR diagnosis. Data were normalized and analyzed to assess the distribution of CD8+ T cell subsets based on transcriptomic gene signatures.

FIG. 11B is a series of violin plots showing the relative expression levels of genes associated with activation, memory, and exhaustion phenotypes. These markers were used to characterize the functional state of CD8+ T cells during rejection in a patient maintained on iscalimab immunosuppressive therapy.

FIG. 12A is a series of UMAP plots illustrating clustering of clonally expanded CD8+ T cells identified by TCR sequencing from the renal biopsy (red dots) and corresponding urine sample (blue dots). The first UMAP (left) displays clonally expanded CD8+ T cells from the biopsy, the second UMAP (middle) shows those from the urine, and the third UMAP (right) overlays clonally expanded CD8+ T cells from both biopsy and urine, highlighting shared clonal populations.

FIG. 12B is a pair of graphs showing the frequency of clonally expanded CD8+ T cell populations in the biopsy compared to the urine.

FIG. 13A is a heatmap of top differentially expressed genes (DEGs) in clonally expanded CD8+ T cells from biopsy and urine samples at the index timepoint, directly compared using supervised analysis. The plot demonstrates significant alignment in gene expression between the two sample types, indicating consistent expression patterns.

FIG. 13B is a violin plot showing the expression of FKBPIA, a gene encoding a tacrolimus binding protein, in clonally expanded CD8+ T cells from biopsy and urine samples at the index timepoint.

### DEFINITIONS

As used herein, the term “about,” when referring to a value or to an amount of mass, weight, time, volume, pH, size, concentration, or percentage, is meant to encompass variations of, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods.

As used herein, the term “ACR” means acute cellular rejection.

As used herein, the term “AMR” means antibody mediated rejection.

As used herein, the term “MAR” means mixed acute rejection.

As used herein, the term “TCR” means T cell receptor for antigen.

As used herein, the term “scRNAseq” means a molecular approach for analyzing gene expression by capturing mRNA from individual cells, barcoding the mRNA in creating cDNA libraries, followed by sequencing and bioinformatics analysis.

As used herein, the term “TCR seq” means a molecular approach by which both TCR alpha and beta chains mRNA are captured, converted to cDNA and sequenced.

### DETAILED DESCRIPTION OF THE INVENTION

One or more specific embodiments of the present invention are described below. In an effort to provide a concise description of these embodiments, all features of an actual implementation may not necessarily be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Rejection therapy has remained unchanged since early reports of high dose corticosteroid use and antilymphocyte globulin use (REFs Starzl) in the 1960's. These empirically-derived anti-rejection therapies, despite relatively poor outcomes in all but Banff 1a ACR episodes, have remained the primary means by which rejection is treated worldwide. As noted above, long term graft survival is poor for many of the clinical phenotypes of rejection. Importantly, the population-based graft survival slope marked increases once rejection has occurred. A major shift in focus in immunosuppressive drug development is needed to mitigate the effects of rejection on long-term allograft survival.

The present invention involves the development of personalized treatments of rejection that are enabled by identification of CD8_{EXP} clonal populations as a therapeutic target. Monitoring of this population for responses to personalized rejection therapy are key to enhancing allograft survival and mitigating allograft loss rates. Therefore, the present invention takes the novel approach of using the gene expression/cell signaling pathways of CD8_{EXP} T cell clonal populations for selection of personalized therapies to treat rejection events. This approach can not only be used to inform potential personalized treatments for rejection, it can also be used to monitor the response to a given therapy.

### Current and Potential Antirejection Treatments

Currently, a number of FDA approved agents exist that may be useful as either primary anti-rejection agents, or as adjunctive agents to corticosteroids and or rATG, or as components in multidrug regimens. As an example, we have identified that some, but not all patients with acute rejection have a substantial population of proliferating CD8 cells. If scRNAseq were to reveal such a population, particularly if it was a CD8_{EXP} clonal population, then potent antiproliferative agents may be useful (examples might include cytostatic drugs such as MMF or mTOR inhibitors, or potential cytocidal drugs such as corticosteroids or proteasome inhibitors). Alternatively, if CD8_{EXP} clonal populations demonstrate marked upregulation of calcineurin inhibitor pathways or increased intracellular receptor expression for tacrolimus-binding proteins, or mTOR pathway components, then selected inhibitors may be used. Importantly, our data have provided proof-of-concept data for the latter example. We found that CD8_{EXP} cells from patients with belatacept-refractory rejection (BRR) have increased expression of mTOR signaling components RPTOR, MTOR, and RCTOR (REF), which validated our prior work showing that patients undergoing BRR can be successfully treated with mTOR inhibitors (REF). Thus, as CD8⁺ T cells are the main drivers of ACR, greater understanding of their gene expression will inform their biology (e.g. proliferation status, effector function) which can be exploited to inform potential treatment options as well as being monitored to define the success of those treatments.

### Inventive Embodiments

In one embodiment, the present invention involves a method for detecting rejection, monitoring for rejection, and monitoring rejection therapy, either invasively (allograft biopsy) or noninvasively (urine sample). The approach of the present invention can also be extended to identify specific cell populations that are driving rejection and determining how they respond to treatment, and also for identifying mechanistically driven novel treatments to improve rejection treatment and outcomes.

In another embodiment, an inventive method is disclosed that involves several steps. First, for an allograft biopsy, the biopsy is first digested using cold protease-based digestion at 4 degrees centigrade to minimized digestion temperature-based gene expression artifacts. If urine is to be used, the urinary sediment is isolated by centrifugation and resuspended in defined buffer solution for performance of scRNAseq. Using the 10x genomics platform with or without TCR or BCR 5′ kits, cDNA libraries are then created in which each individual mRNA molecule is encoded with information relating to its cell of origin. The library is then sequenced and made available for bioinformatics analysis. Bioinformatics analysis is performed using Cell Ranger and Seurat and individual cell populations are annotated. Additionally, the cells are phenotyped based on gene expression patterns. If 5′ based kit has been used, T cell (or B cell) clonal populations are defined. Expanded T and B cell populations are identified using either TCR or BCR sequencing, which will enable identification of expanded clonal populations, which is indicative of expansion of allo-reactive cells. These expanded clonal populations are then analyzed in detail to define gene expression, particularly for genes that are involved in cellular-based attacks on the patient's transplanted organ. Examples of markers that may further identify individual cells within the expanded populations include CD8⁺ T cells that express effector molecules such as granzymes and perforin. Degranulation markers (CD107a) can also be utilized to define cells that have recently mediated allograft injury via granzyme and perforin release. Alloreactive CD8⁺ T cells (and possibly in some patients CD4⁺ T cells and also potentially gamma delta T cells) can also be further identified by activation markers including expression of HLA antigens, cytokine receptors, and degranulation markers as noted above.

Once these cells are identified, extensive transcriptomic profiling is used to determine signaling pathways that can then be analyzed using pathway identification software. Selected pathways can then be targeted by FDA approved drugs of a number of classes including as examples tyrosine kinase inhibitors, antiproliferative agents (eg MMF), and possible depleting/blocking agents including monoclonal antibodies. Because these cells can be uniquely identified by their TCR genes, our published data show that these cells can then be monitored serially either invasively by biopsy or noninvasively via urine to determine if these cell populations continue to exist or have expanded, or if they have been reduced or eliminated. Elimination of these T cell clones has been associated with rejection reversal in our experience to date. These new targeted therapies can be used alone or in combination with other existing potential rejection therapies such as corticosteroids or antilymphocyte antibodies.

### Single Cell RNASeq

In one embodiment, the present invention involves a technique for assessing rejection using an innovative, highly powerful multiplexed technology termed single cell RNASeq (scRNAseq). This technique provides data regarding gene expression in thousands of cells derived either from urinary sediment or allograft biopsy in kidney transplant recipients. This technique uses single cell suspensions for analysis. In the case of a renal allograft biopsy, the technique involves tissue digestion with selected enzymes, preferably a set of enzymes selected for their ability to digest tissues at 4 degrees centigrade (cold digestion proteases). Cold digestion has been shown to reduce temperature-induced artifacts associated with tissue digestions at 37 degrees centigrade (REF). The technique has been modified to enable digestion of either fresh or frozen tissues.

Once the cell preparation is complete, scRNAseq analysis is performed, currently on the 10x Genomics platform, although the protocol can be adapted for other platforms. TCR seq is essential for identifying expanded CD8 clones (CD8EXP), and for this a 5′ TCR kit is used. scRNAseq process will generate a cDNA library, which is then sequenced using any of a variety of platforms. Once sequencing is complete, the data set is then available for analysis.

Bioinformatic analysis can be performed using a variety of software packages and pipelines. Currently, Cell Ranger is utilized for QA assessment, which is followed by Seurat which is used for annotation and analysis of gene expression.

Data derived from scRNAseq will generate information on a significant number of immune cell populations (eg, CD8 T cells, CD4 T cells, macrophage, NK, B cell, neutrophils, et al) and also kidney-derived cell populations (proximal and distal tubular cells, collecting duct cells, ascending loop of Henle, thick ascending limb, podocytes, endothelial cells, et al). Ongoing and future analyses are determining whether these cell populations can add to rejection diagnosis efficiency.

An important consideration for the present invention is our demonstration that CD8_{EXP} clonal populations are expanded because they are alloreactive, and as such, represent the terminal effector cells in cellular rejection. By identifying these highly selective cell populations, they can then be examined for prominent signaling pathways as a means for selecting therapeutic approaches, primarily by using targeted drugs. Examples of pathways that we have identified thus far are CD8_{EXP} memory cell populations in rejection occurring under belatacept-based immunosuppression. In these CD8_{EXP} memory cell populations, we have found mTOR pathway upregulation and have therefore used mTOR inhibitors with success in treating these rejections (REFs). Similarly, in rejection occurring under CD40 blockade (with iscalimab) upregulation of tacrolimus binding proteins (FKBPs) has led us to use short-term add on tacrolimus, which has provided effective rejection treatment, thereby obviating the need for nonselective intensive immunosuppression with depleting rATG therapy.

Bioinformatic analysis for selecting optimal rejection therapies may be supervised or unsupervised. Pipeline analysis of known signaling pathways provides a nonbiased approach for determining potential therapeutics that will constrain the CD8_{EXP} clonal populations that are driving the rejection. Since many therapeutics exist for numerous pathways involved in specific cancers, these same therapeutics may be used for specifically targeted CD8_{EXP} clonal populations.

Given that the approach of the present invention allows detection of the primary effector cells in rejection, the approach of identifying CD8_{EXP} may also be used to diagnose or detect rejection by serial monitoring of either the allograft or the urine. Indeed, the data presented herein shows a strong correlation between CD8_{EXP} clonal populations in the allograft and in the urine, thereby enabling noninvasive assessment of the presence of rejection by scRNAseq analysis of urinary specimens. In an analogous manner, once rejection has been diagnosed, scRNAseq analysis of urinary specimens can also be used to assess the response to therapy noninvasively, and even to alter therapy based on analysis of current CD8EXP clonal populations.

### Figures

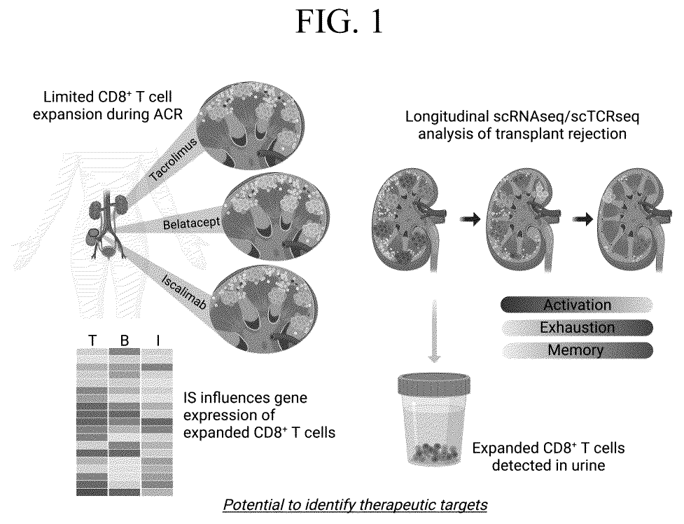

FIG. 1 depicts results from scRNAseq analysis of ten renal allograft biopsies including three biopsies from patients on tacrolimus-based immunosuppression who were not experiencing rejection, and four from patients on tacrolimus-based immunosuppression that were experiencing rejection, three from patients on belatacept (CD28 blockade) based immunosuppression that were experiencing rejection, and three from patients on iscalimab (CD40 blockade) based immunosuppression that were experiencing rejection.

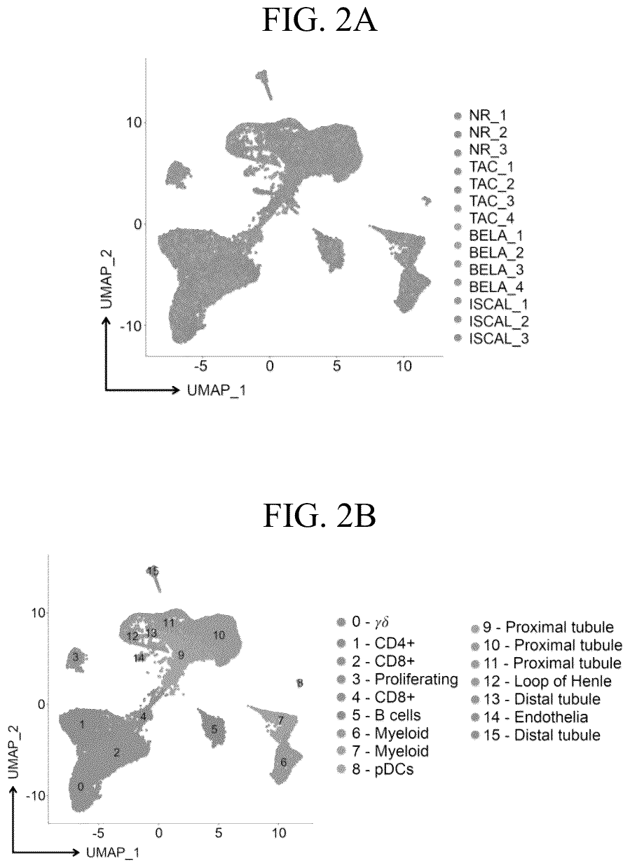

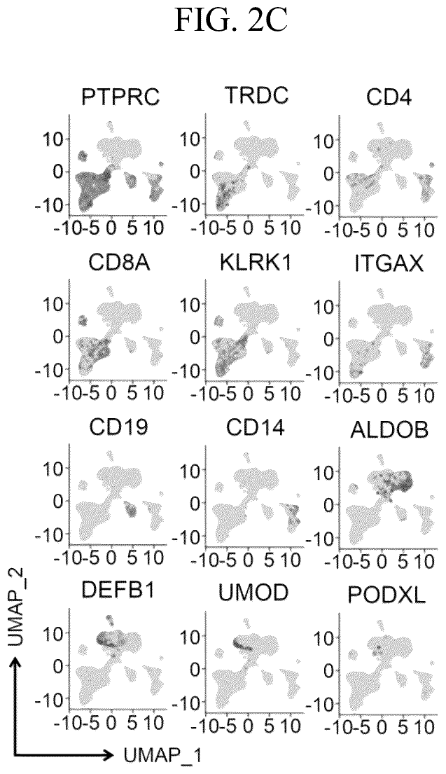

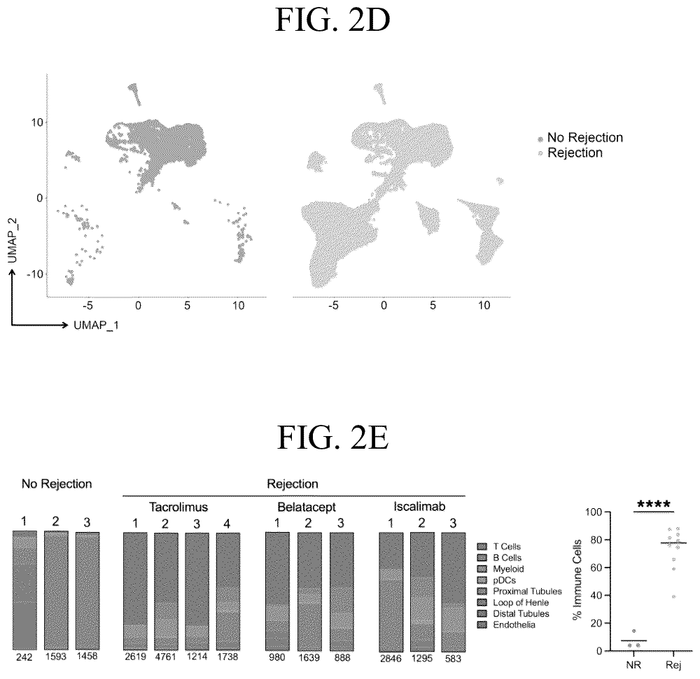

FIGS. 2A-2E show a scRNA-Seq analysis of transplanted kidney allografts. Single-cell suspensions from 13 different biopsies (3 without rejection, 10 with rejection) were individually subjected to 5′ scRNA-Seq on the 10x platform with V(D)J sequencing. After alignment using Cell Ranger, cells with more than 25% mitochondrial content and less than 200 genes, including additional low-quality cells, were removed, and samples were integrated using Seurat.

FIGS. 2A and 2B present UMAP representations of individual cell populations derived from gene expression data including 8 general types of immune cell populations and 7 types of nephron-derived cell populations. FIG. 2C depicts genes used for annotation. FIG. 2D depicts the marked increase in inflammatory cell populations in the presence of acute rejection. FIG. 2E provides a bar graph representation of individual cell populations in the absence of rejection, and the presence of rejection under varying types of maintenance immunosuppression.

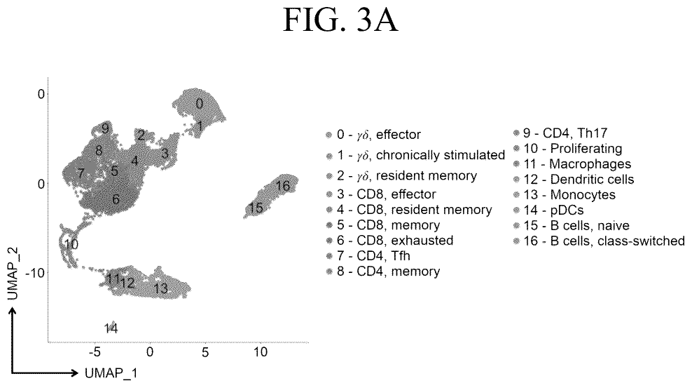

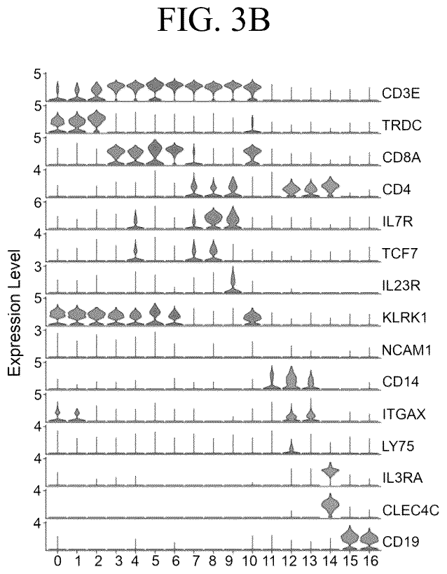

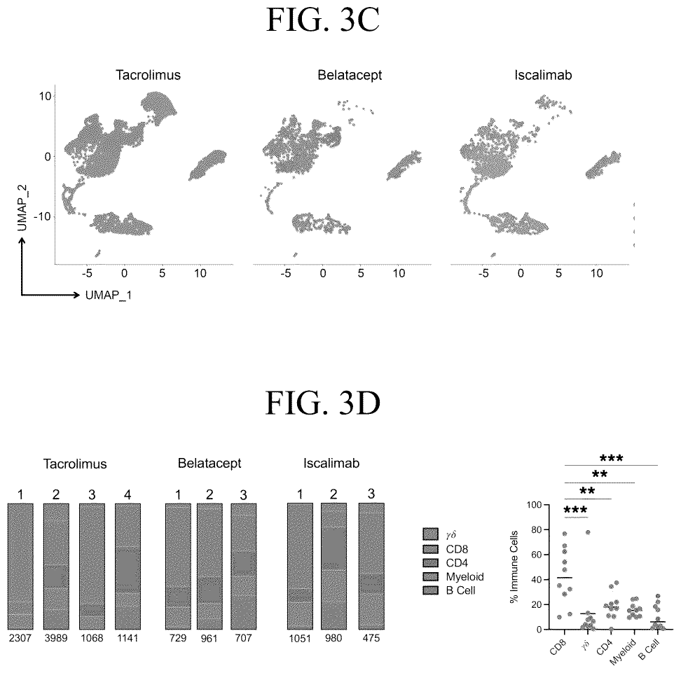

FIGS. 3A-3D present detailed data on individual inflammatory cell populations including gene expression data within these cell populations during acute rejection occurring under varying types of immunosuppression. FIGS. 3A-3D are a series of images showing that diverse immune cells infiltrate during kidney allograft rejection. Index samples from the 10 participants undergoing rejection were integrated, clusters annotated as nonimmune cells were removed, and the data were renormalized and reclustered using Seurat.

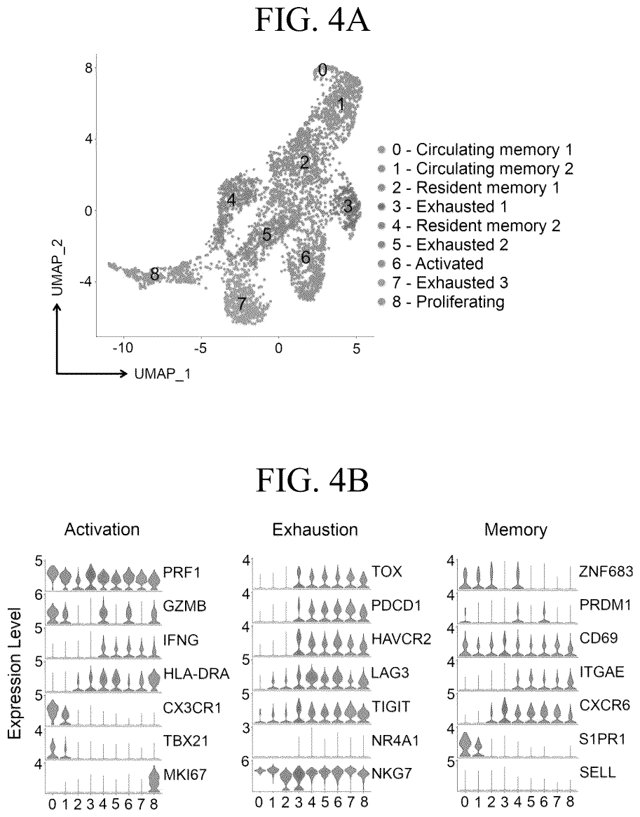

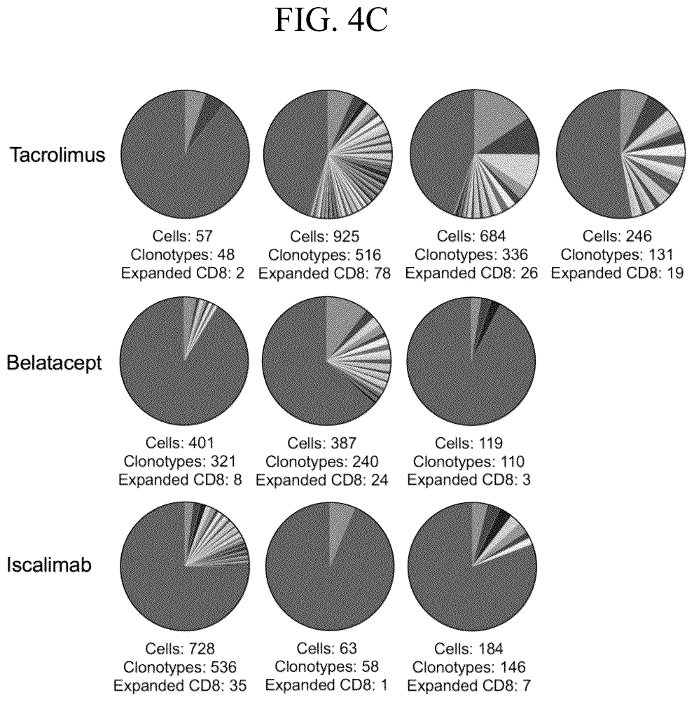

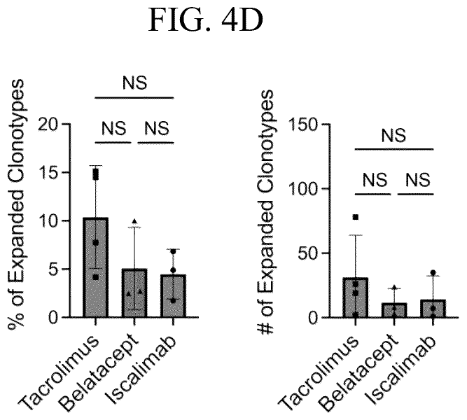

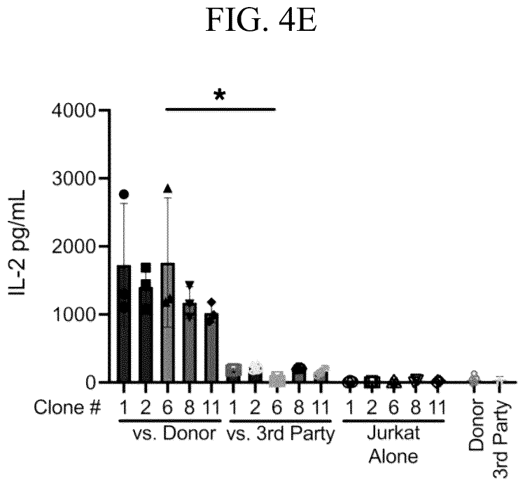

FIG. 4A-4E present analysis of infiltrating CD8⁺ cell populations during acute rejection, including individual gene expression levels for specific gene sets associated with activation, exhaustion and memory phenotypes. FIGS. 4A-4E are a series of images showing analysis of infiltrating CD8⁺ T cells in kidney allograft rejection. CD8⁺ clusters from the immune-cell analysis were identified for further analyses; CD4⁺ and γ/δ T cells were removed. The samples were then reanalyzed using Seurat. FIG. 4C presents data on individual CD8⁺ T cell clonal populations, depicting expanded (colored slices) and nonexpanded (gray) populations under the three types of maintenance immunosuppression. FIG. 4E presents data demonstrating the ability of transfer of individual TCR alpha and beta genes to confer alloreactivity to a TCR negative Jurkat variant cell line.

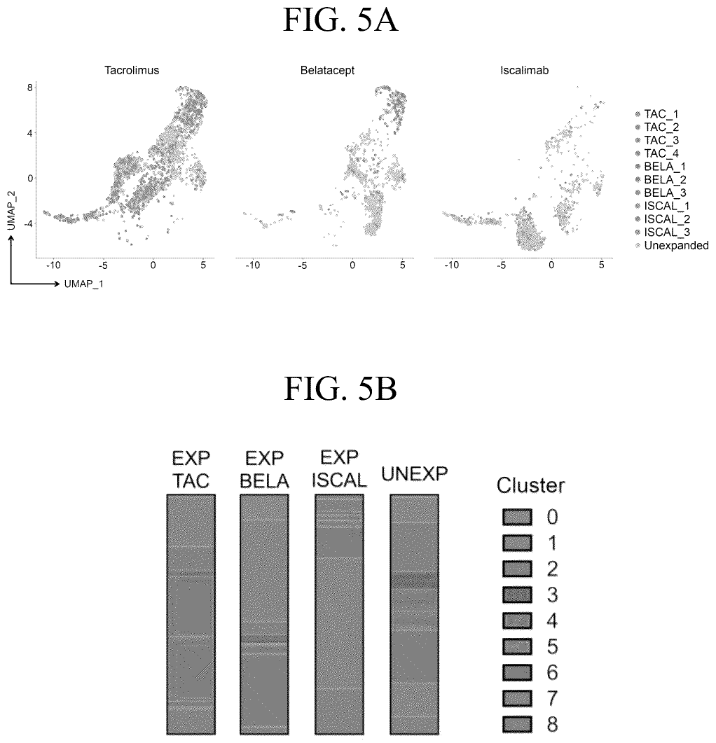

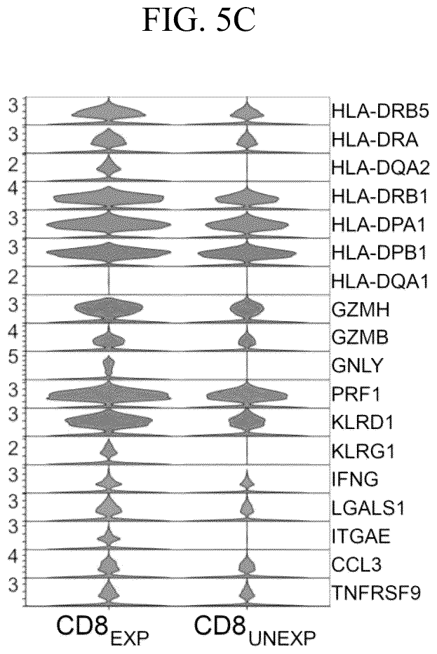

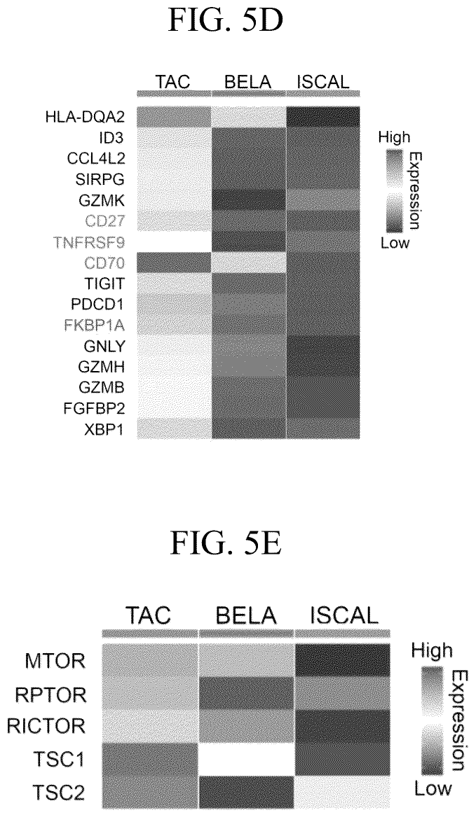

FIGS. 5A-5E present data on clonally expanded CD8⁺ cell populations (CD8_{EXP}) under varying types of immunosuppression, along with supervised and unsupervised analyses of expression of individual genes within CD8_{EXP} and CD8_{UNEXP} cell populations. FIGS. 5A-5E are a series of images showing gene expression differences in CD8_{EXP} among tacrolimus, belatacept, iscalimab maintenance IS. FIGS. 5D and 5E demonstrate how expression of defined genes may be utilized to select individual (ie, personalized) targeted therapies for rejection.

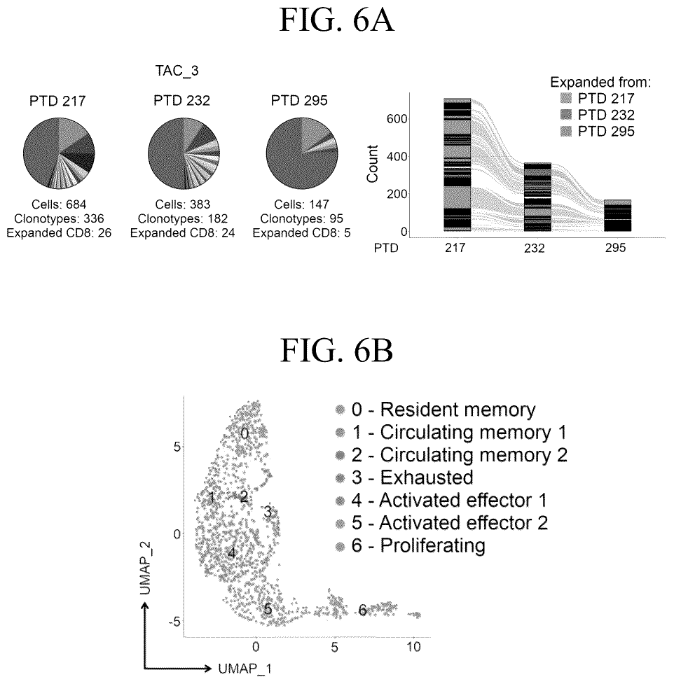

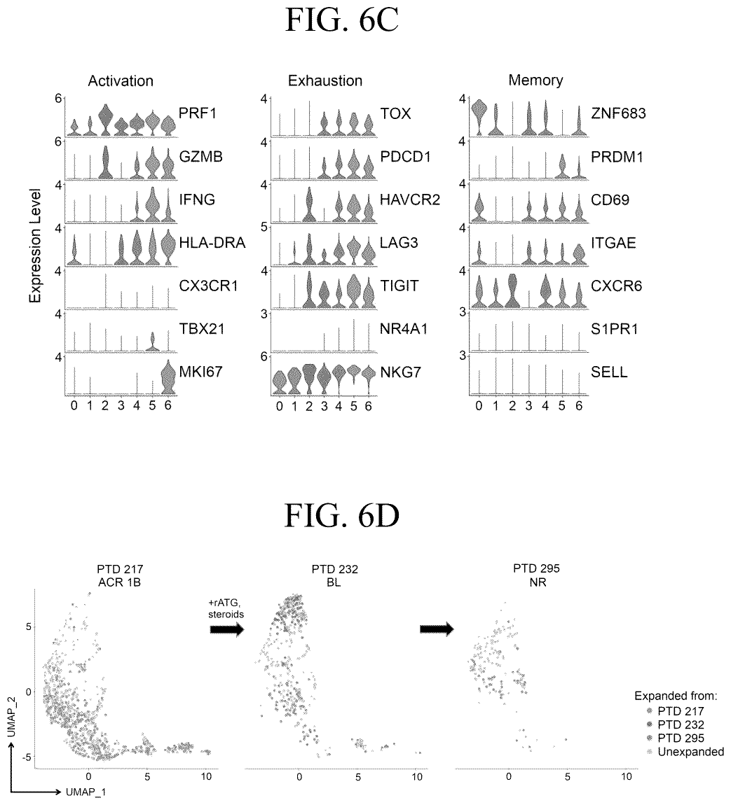

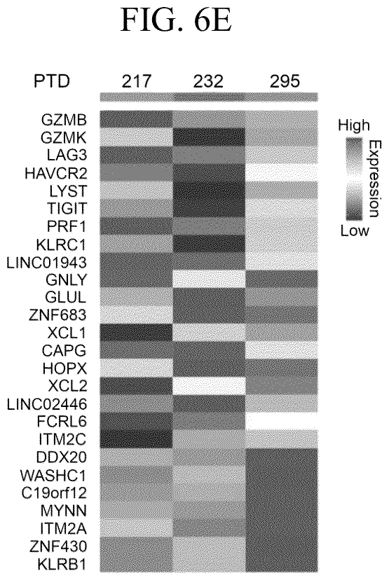

FIGS. 6A-6E present CD8_{EXP} clonal population data including gene expression data at three consecutive time points in an individual patient experiencing acute rejection under tacrolimus-based immunosuppression. The data show temporal scRNA-Seq analysis of the response to antirejection therapy under tacrolimus maintenance IS. A participant on tacrolimus IS (TAC_3) was diagnosed with ACR 1B on PTD 217, and a biopsy was obtained prior to antirejection treatment with rATG and steroids. A second biopsy was obtained on PTD 232, and the participant was diagnosed with a borderline lesion. A third biopsy was taken at PTD 295, and the participant was diagnosed with no rejection.

These data demonstrate how scRNAseq data can be used to specifically analyze CD8_{EXP} clonal population responses to rejection therapy, and how these populations may expand or contract with rejection treatment, and also how individual cell populations may adapt to individual types of rejection therapy. In this manner, one can utilize CD8_{EXP} clonal population data to select rejection therapies and also monitor response to rejection therapies over time.

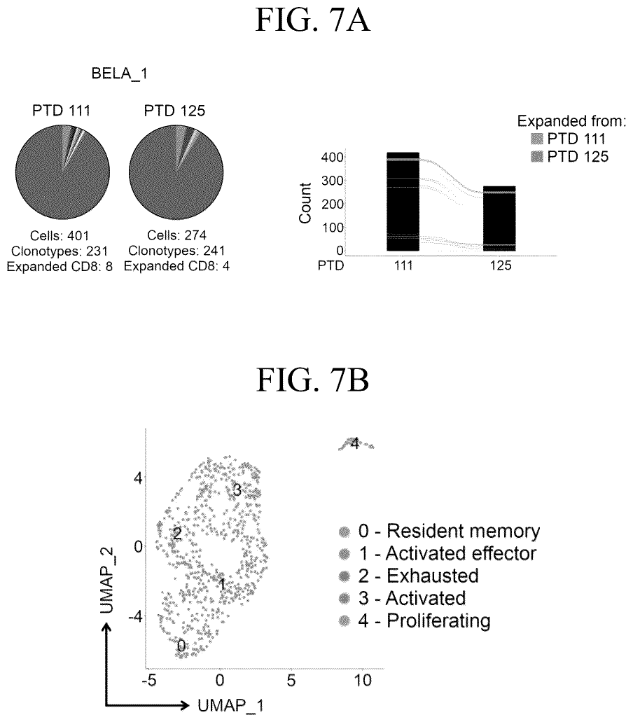

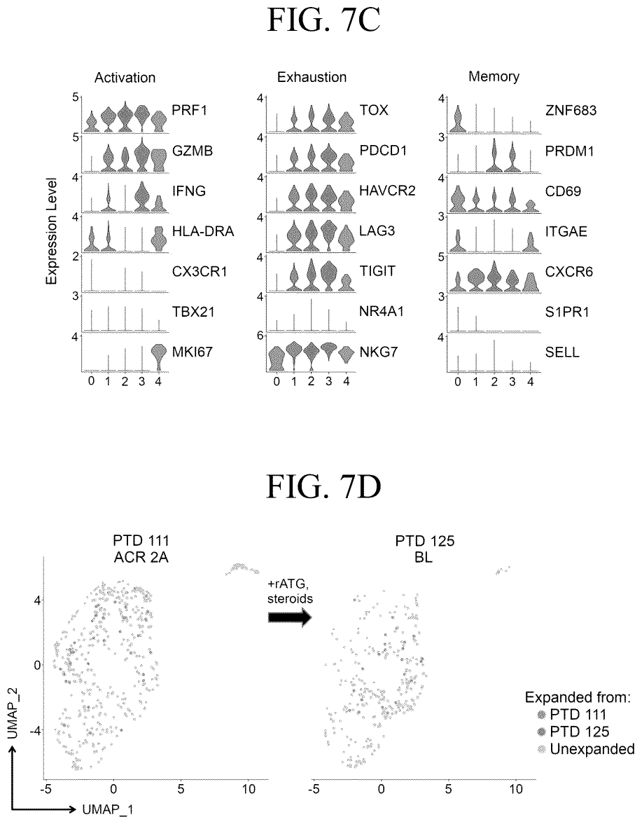

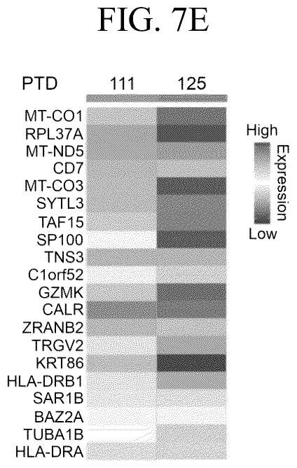

FIGS. 7A-7E present scRNAseq data that is similar to data presented on CD8_{EXP} in FIG. 6 in another patient being treated for rejection that occurred under belatacept-based immunosuppression. The data shows temporal scRNA-Seq analysis of the response to antirejection therapy under belatacept maintenance IS. A participant on belatacept IS (BELA_1) was diagnosed with ACR 2A on PTD 111, and a biopsy was obtained prior to antirejection treatment with rATG and steroids. A second biopsy was obtained on PTD 125, and the participant was diagnosed with a borderline lesion.

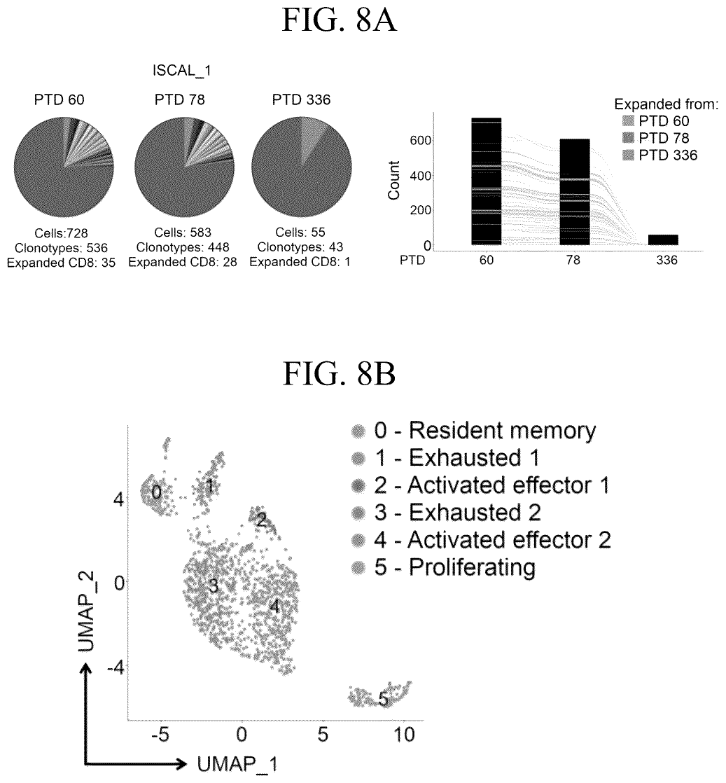

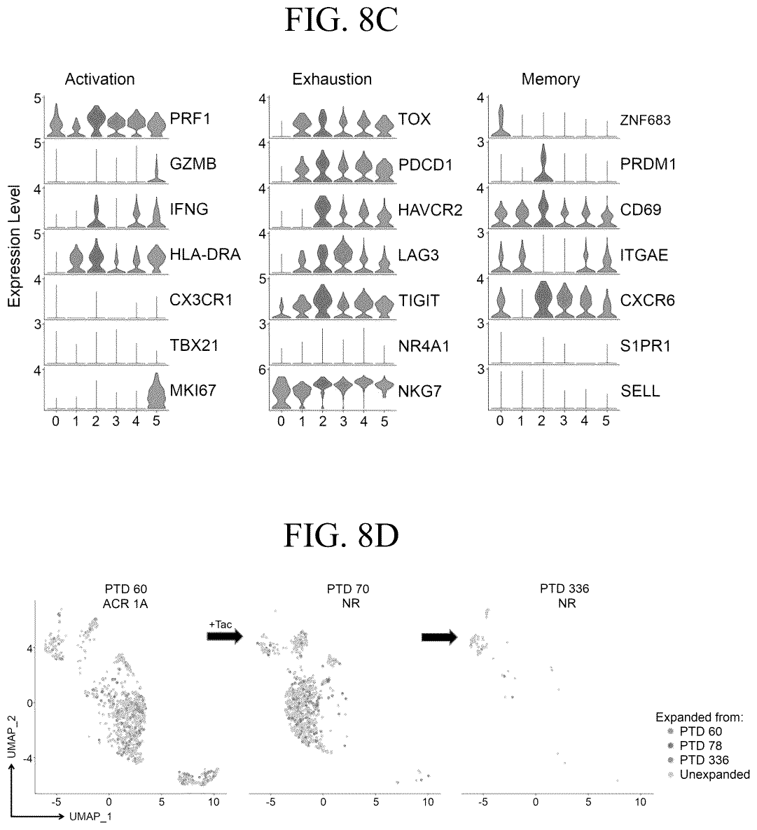

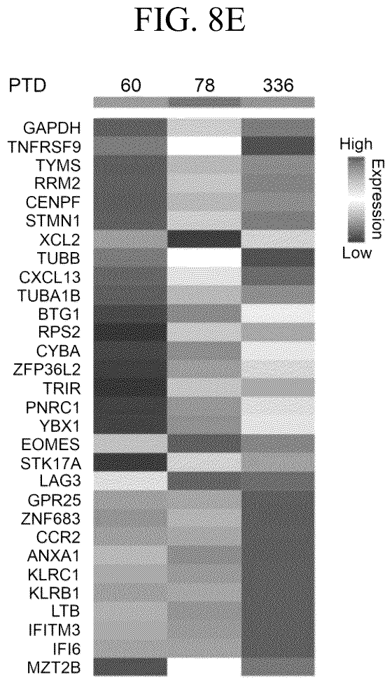

FIGS. 8A-8E present scRNAseq data similar to data presented on CD8_{EXP} in FIGS. 6 and 7 above in a third patient being treated for rejection, with this patient experiencing rejection while on iscalimab based immunosuppression. The data show temporal scRNA-Seq analysis of the response to antirejection therapy under iscalimab maintenance IS. A participant on iscalimab IS (ISCAL_1) was diagnosed with ACR 1A on PTD 60, and a biopsy was obtained prior to antirejection treatment with tacrolimus. A second biopsy was obtained on PTD 78, and the participant was diagnosed with no rejection. A third biopsy was taken at PTD 336, and the participant was again diagnosed with no rejection. These data demonstrate how short-term (10 days) tacrolimus therapy (selected as a therapy based on CD8_{EXP} gene expression data) can markedly alter CD8_{EXP} gene expression with eventual near elimination of CD8_{EXP} clonal populations.

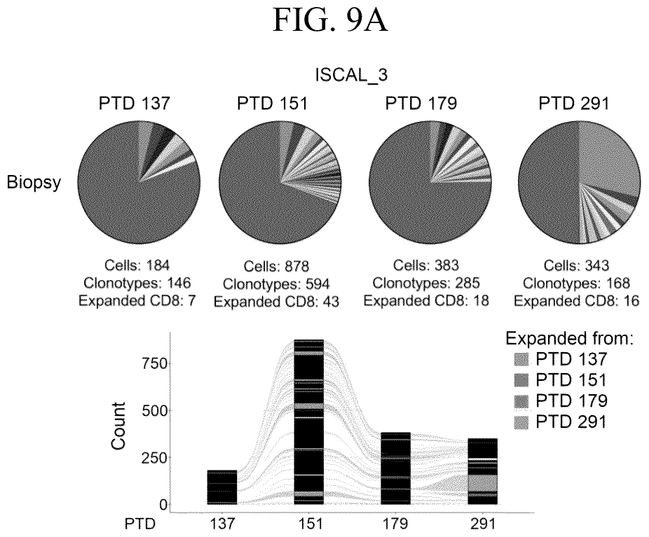

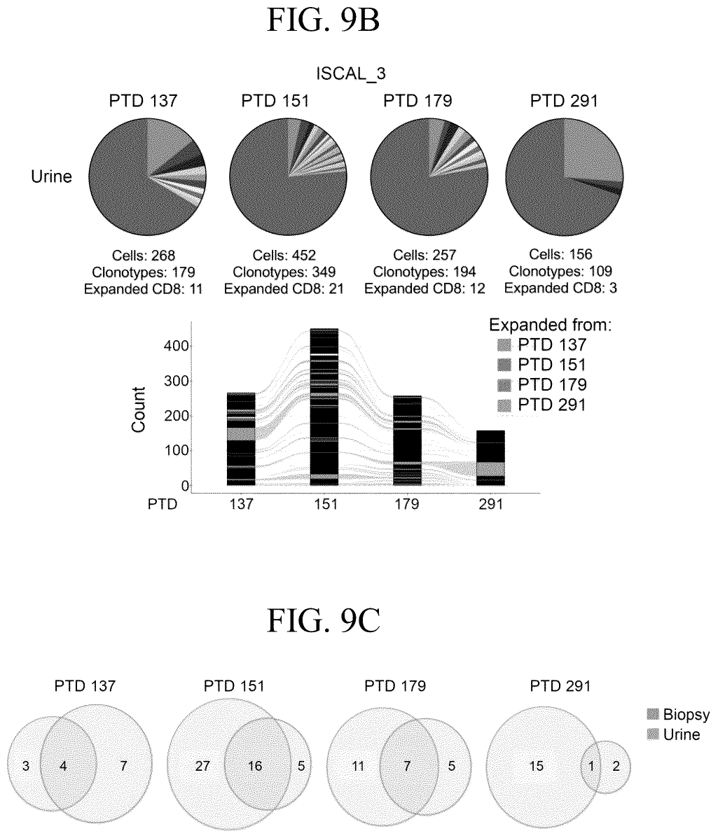

FIGS. 9A-9C present CD8_{EXP} clonal population data from paired urine and allograft biopsy samples demonstrating the remarkable degree of symmetry between urine and allograft, indicating that the urine can be used as a noninvasive surrogate for allograft biopsy-based assessment of CD8_{EXP} clonal populations. The data shows a comparison of CD8_{EXP} between the biopsy and paired urine samples in a participant undergoing treatment-refractory rejection. A participant on iscalimab IS (ISCAL_3) was diagnosed with ACR 1B on PTD 137, and a biopsy was obtained prior to antirejection treatment with tacrolimus conversion and steroids. A second biopsy was obtained on PTD 151, and the participant was diagnosed with ACR 1B. MMF was then added to the antirejection regimen. A third biopsy was taken at PTD 179, and the participant was diagnosed as borderline and MMF was tapered off. A final biopsy was taken at PTD 291 and showed mixed 1B rejection.

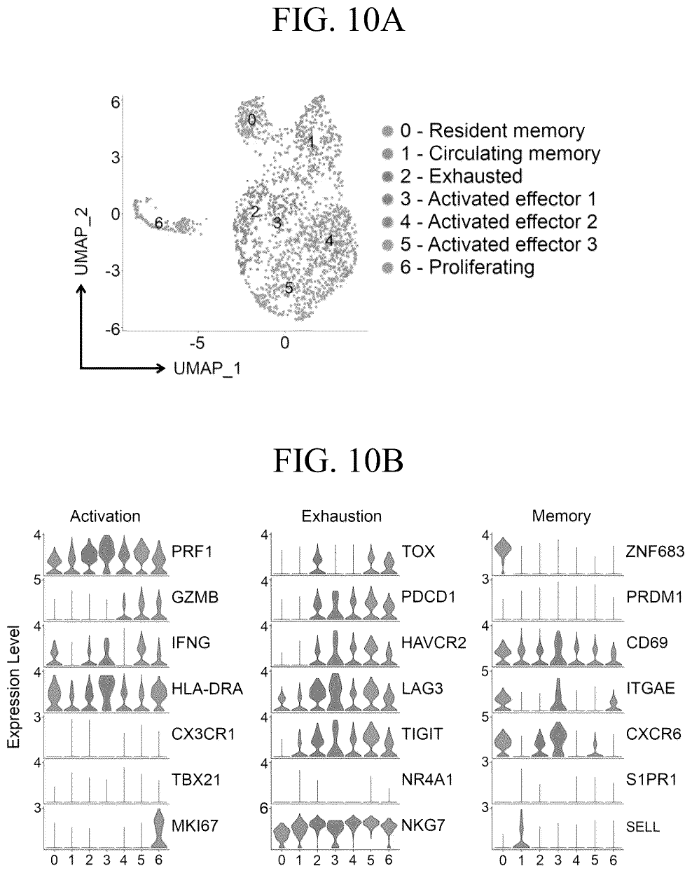

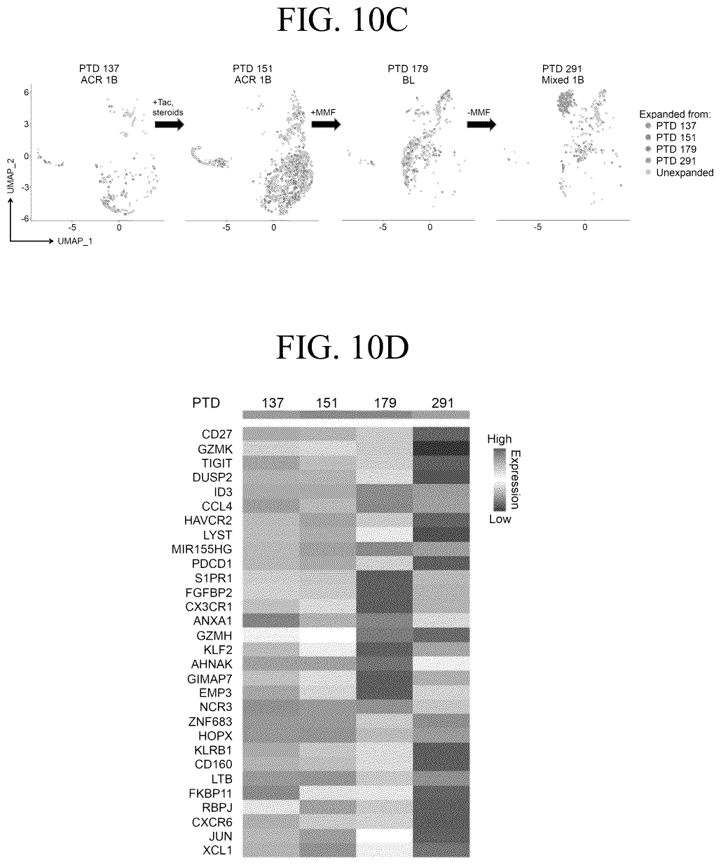

FIGS. 10A-10D present scRNAseq data on T cell populations and CD8_{EXP} clonal populations in a patient with refractory acute cellular rejection with four serial biopsies obtained over a few months period of time. Note the remarkable changes in CD8_{EXP} clonal populations over time and the varying responses to individual rejection treatment strategies.

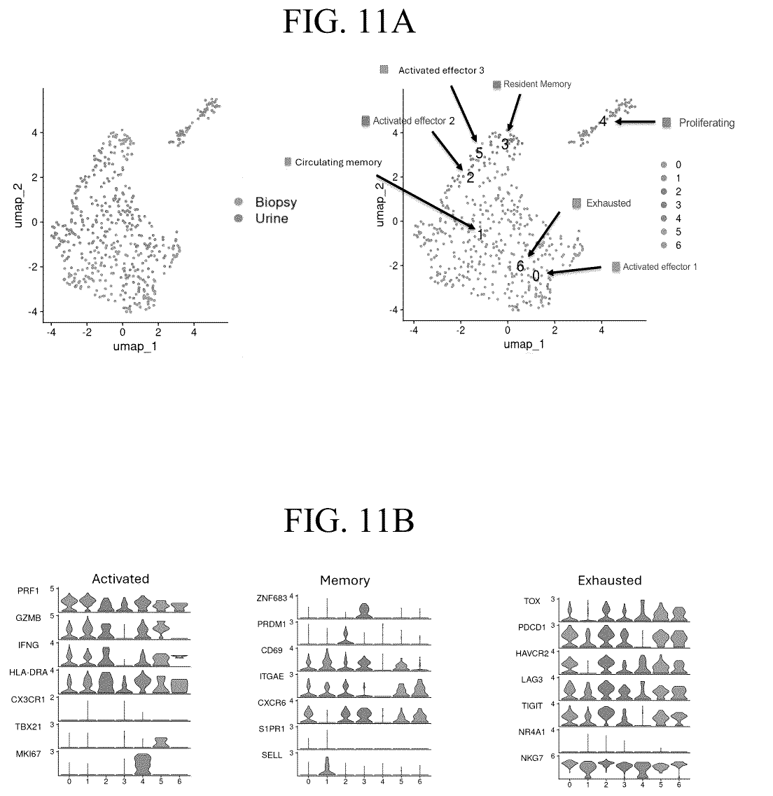

FIGS. 11A and 11B show an analysis of CD8+ T cells from matched renal biopsy and urine samples at the index timepoint of ACR diagnosis (Banff score of 1B) using scRNAseq.

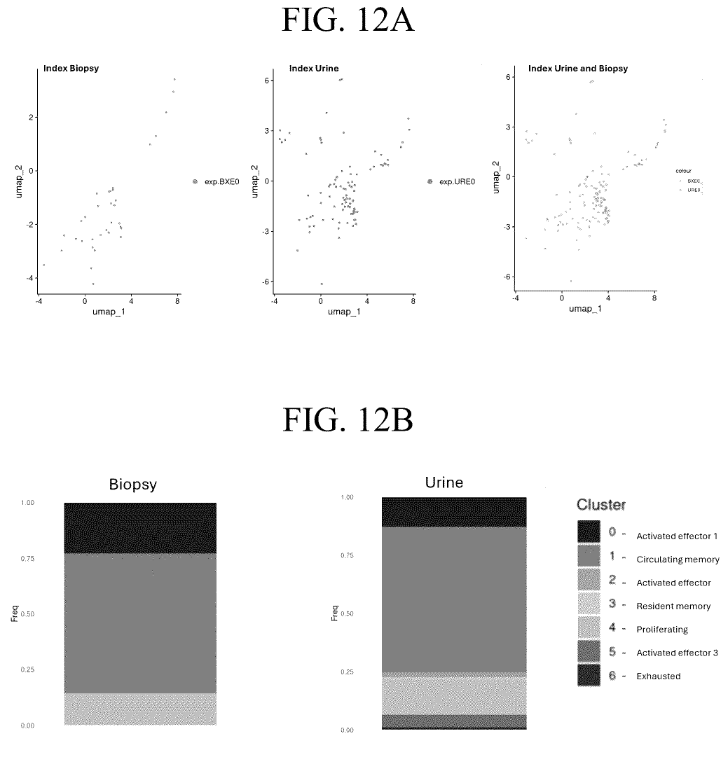

FIGS. 12A and 12B show similar phenotypes of clonally expanded CD8+ T cells in matched biopsy and urine samples at ACR diagnosis.

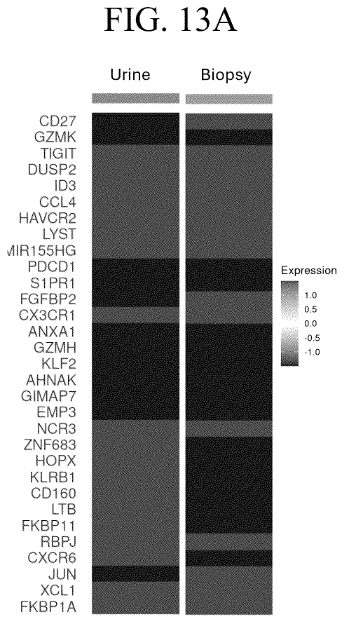

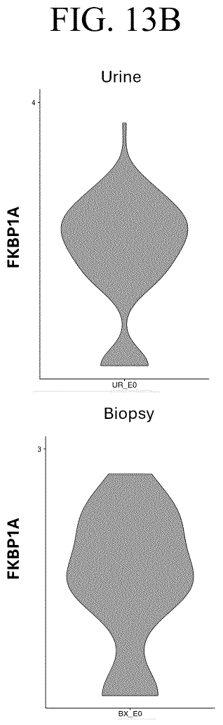

FIGS. 13A and 13B show similar gene expression between clonally expanded CD8+ T cells in urine and biopsy at the time of ACR diagnosis.

### EXAMPLES

### Example 1: Rejection Diagnosis

FIGS. 4A-4E present ten cases of acute cellular rejection where CD8_{EXP} T cell clones were identified, thereby indicative of acute rejections.

### Example 2: Treatment Selection

FIGS. 5A-5E, 6A-6E, 7A-7E, 8A-8E and 10A-10D demonstrate how gene expression patterns in CD8_{EXP} T cell clonal populations may be utilized to select therapeutic agents.

### Example 3: Monitoring Rejection Treatment

FIGS. 5A-5E, 6A-6E, 7A-7E, 8A-8E and 10A-10D demonstrate how gene expression patterns in CD8_{EXP} T cell clonal populations may be utilized to monitor rejection treatment by performance of serial scRNAseq/TCR analysis of specimens. These figures also demonstrate contraction and expansion or stability of individual CD8_{EXP} T cell clonal populations which provides information on response to rejection therapy.

While all the invention has been illustrated by a description of various embodiments, and while these embodiments have been described in considerable detail, it is not the intention of the Applicant to restrict or in any way limit the scope of the appended claims to such detail. Additional advantages and modifications will readily appear to those skilled in the art. The invention in its broader aspects is therefore not limited to the specific details, representative apparatus and method, and illustrative examples shown and described. Accordingly, departures may be made from such details without departing from the spirit or scope of the Applicant's general inventive concept.

What is claimed is:

1. A method for performing an analysis related to potential rejection of an organ after transplantation in a subject, the method comprising identifying alloreactive/expanded CD8 T cell clones in either an allograft biopsy or a urine sample from the subject, wherein the T cell clones are identified using a single cell RNASeq (scRNAseq)/TCRseq test.

2. The method of claim 1, wherein the analysis is selected from the group consisting of detection of rejection, diagnosis of rejection, treatment selection, therapeutic monitoring for rejection, identification of resistance to rejection treatment, and combinations thereof.

3. The method of claim 2 wherein alloreactive/expanded CD8 T cell clones are identified from an allograft biopsy.

4. The method of claim 2 wherein alloreactive/expanded CD8 T cell clones are identified from a urine sample.

5. The method of claim 2 wherein the analysis comprises detecting rejection of a transplanted organ in a subject.

6. The method of claim 2 wherein the analysis comprises monitoring for rejection of a transplanted organ in a subject.

7. The method of claim 2 wherein the analysis comprises monitoring rejection therapy.

8. The method of claim 1, wherein the organ is a kidney.

9. The method of claim 8, wherein the allograft biopsy is a renal allograft biopsy.

10. The method of claim 9, wherein the renal allograft biopsy involves tissue digestion with cold digestion proteases.

11. A method for assessing responses of a subject to anti-rejection therapies, the method comprising identifying alloreactive/expanded CD8 T cell clones in either an allograft biopsy or a urine sample from the subject, wherein the T cell clones are identified using a single cell RNASeq (scRNAseq)/TCRseq test.

12. A method for identifying specific cell populations that are causing rejection of a transplanted organ in a subject, the method comprising identifying alloreactive/expanded CD8 T cell clones in either an allograft biopsy or a urine sample from the subject, wherein the T cell clones are identified using a single cell RNASeq (scRNAseq)/TCRseq test.

13. The method of claim 12 wherein the specific cell populations are CD8+ T cells that express effector molecules.

14. The method of claim 13 wherein the CD8+ T cells are selected from the group consisting of granzymes, perforin and combinations thereof.

15. The method of claim 12, the method further comprising using transcriptomic profiling on the specific cell populations to determine signaling pathways and analyzing the signaling pathways using pathway identification software.
